(19) United States
(12) Patent Application Publication
(10) Pub. No.: US 2018/0333573 A1
(43) Pub. Date: Nov. 22, 2018

(54) PROTECTION OF BIOLOGICAL SYSTEMS

(71) Applicant: Frederick Stephen Felt, Provo, UT (US)

(72) Inventor: Frederick Stephen Felt, Provo, UT (US)

(21) Appl. No.: 15/982,668

(22) Filed: May 17, 2018

Related U.S. Application Data
(60) Provisional application No. 62/603,117, filed on May 18, 2017.

(51) Int. Cl. A61N 1/14; A41D 13/008

(52) U.S. Cl. CPC A61N 1/14; H05F 3/025; A41D 13/008

(57) ABSTRACT

An apparatus, system, and method are disclosed for protecting a biological system from molecular damage due to electrostatic discharge. The apparatus, system, and method include an electrical contact point, a resistive element, and a connection point. The electrical contact point is positioned to facilitate electrical communication with the biological system. The resistive element is coupled to the electrical contact point. The resistive element has an electrical resistive value tuned to drain electrostatic charge from the biological system based on a contact time of the electrical contact point with the biological system. The connection point is coupled to the resistive element to create an electrical potential difference across the resistive element in response to the contact of the electrical contact point with the biological system.

### CROSS-REFERENCES TO RELATED APPLICATIONS

This application claims the benefit of U.S. Provisional Patent Application No. 62/603,117 entitled “Use of antistatic materials and electrical personnel grounding equipment to protect DNA from damage or mutation by ESD (Electrostatic Discharge) in order to prevent cancer and human aging” and filed on 18-May-2017 for Frederick Stephen Felt, the entire contents of which are incorporated herein by reference for all purposes.

### FIELD

This invention relates to electro-static discharge (ESD) protection and more particularly relates to protection of biological systems.

### BACKGROUND

Electro-static charge imbalances may be induced when an object makes contact, or proximity, with and then separates from another object with one or more of the objects being electrically insulating. Electro-static charge may also be induced by proximity to an object or surface. Electro-static charge may also be induced by pressure, heating, and proximity to another charged object. The charge is then neutralized when the charged object comes into electrical communication with a conductive or grounded object. The neutralization of the charge is an event known as an electro-static discharge (ESD). Additionally, ESD events may be sustained by typically insulating materials upon exceeding a breakdown voltage of the material.

### SUMMARY

An apparatus for protect a biological system from molecular damage due to electrostatic discharge is disclosed. The apparatus includes an electrical contact point, a resistive element, and a connection point. The electrical contact point is positioned to facilitate electrical communication with the biological system. The resistive element is coupled to the electrical contact point. The resistive element has an electrical resistive value tuned to drain electrostatic charge from the biological system based on a contact time of the electrical contact point with the biological system. The connection point is coupled to the resistive element to create an electrical potential difference across the resistive element in response to the contact of the electrical contact point with the biological system

A method for protecting a biological system from electrostatic discharge damage is also disclosed. The method includes determining a contact time for the biological system at an electrical contact point. The method also includes coupling a resistive element to the electrical contact point. The resistive element has a resistive value tuned, based on the contact time, to drain electrostatic charge from the biological system. The method also includes coupling a connection point to the resistive element to create an electrical potential difference across the resistive element in response to contact of the biological system with the electrical contact point.

A wearable system to protect a biological system from molecular damage due to electrostatic discharge is also disclosed. The wearable system includes an electrical contact point, a resistive element, a connection point, and a tracking system. The electrical contact point is positioned to facilitate electrical communication with the biological system. The resistive element is coupled to the electrical contact point. The resistive element has an electrical resistive value tuned to drain electrostatic charge from the biological system based on a contact time of the electrical contact point with the biological system. The connection point is coupled to the resistive element to create an electrical potential difference across the resistive element in response to the contact of the electrical contact point with the biological system. The tracking system tracks data corresponding to the biological system.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention, and are not therefore to be considered to be limiting of its scope, the invention will be described and explained with additional specificity and detail through the use of the accompanying drawings, in which:

FIG. 1 is a perspective view illustrating one embodiment of a DNA protection system 102 in accordance with the present invention;

FIG. 2 is an enlarged view illustrating one embodiment of a DNA protective apparatus 200 local to a biological system 104 in accordance with the present disclosure;

FIG. 3 is a diagram illustrating different environments in which embodiments of DNA protection may be implemented in accordance with the present disclosure; and

FIG. 4 is a flowchart diagram illustrating one embodiment of a method 400 for protecting DNA from electrostatic discharge damage in accordance with the present disclosure.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. The term “point” means a single point, multiple points, a region, or area unless expressly specified otherwise.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

These features and advantages of the embodiments will become more fully apparent from the following description and appended claims or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods, and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

ESD events have been recognized as destructive for sensitive electronic microcircuits but, until now, little has been understood about the effect on biological systems. The energy contained in a single ESD event is known to reach relatively high and potentially destructive levels. For example, ESD events may reach 15,000 volts, 25,000 volts, 50,000 volts, or more with a current of 30 amperes or greater. Such levels are considered fatal in a sustained event. It is due to the relatively brief period of time in which these events occur that common ESD events are not fatal. However, the level of energy involved in a common ESD events is above levels capable of causing cellular and molecular damage to biological systems.

A single ultraviolet (UV) photon is known to affect damage on deoxyribonucleic acids (DNA) resulting in mutations that can lead to cancers. The energy of the average UV photon is orders of magnitude less than that of the average ESD event. Therefore, the energy contained in each ESD event is more than sufficient to cause fundamental damage to DNA. While most damage is repaired by enzymes, with frequent and repeated damage, the chance of persistent damage increases. As ESD events are common and occur in a wide range of situations in daily life, the risk of DNA damage due to ESD is considerable.

The human body may be approximately modeled as a capacitor. The energy of a capacitor may be represented mathematically as E=½CV² where E=energy, C=capacitance, and V=voltage. Because voltage (V) is a squared term in the equation, decreasing the voltage in a human body model results in a large reduction in the energy involved in the system. The term “human body model” refers to a circuit diagram with a behavior approximating or simulating a human body.

Further, because the behavior of a capacitor, and the human body, is asymptotic in nature, the energy never truly reaches zero. Solutions provided herein seek to optimize available contact time to shunt charge in a safe manner to avoid biological damage. For example, mitochondrial and nuclear DNA damage and damage to other cellular components and system due to ESD events are prevented or significantly reduced.

Described herein are methods, systems, and apparatuses which have a tuned resistance value based on an expected time of contact. Embodiments described herein may have a resistive value tuned to draw down the charge built up on a biological system. The amount the charge is drawn down may be the total charge amount or a portion of the total charge. In some embodiments, the tuning may also be based on a frequency of touches as well as a material involved in each touch. For example, a system with more frequent touches with the biological system may have a higher resistance to draw off less charge to lower the amount of energy applied to the DNA with each draw-down. The higher frequency of touches would allow for draw-downs to occur more often which would keep the charge of a biological system at safe levels and reduce damage from discharges.

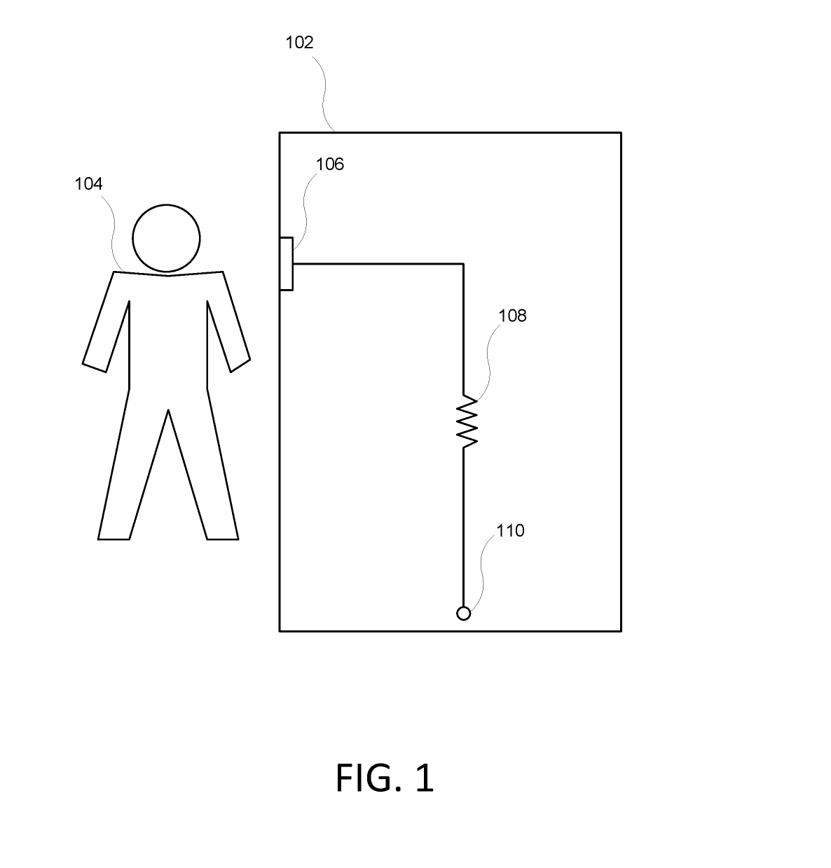

FIG. 1 is a perspective view illustrating one embodiment of a DNA protection system 102 in accordance with the present invention. In the illustrated embodiment, the DNA protective system 102 is remote to a biological system 104. In the illustrated embodiment, the biological system 104 is a person. In other embodiments, the biological system 104 is a non-human system. In the illustrated embodiment, the DNA protection system 102 includes a contact point 106, a resistive element 108, and a connection point 110.

In some embodiments, the contact point 106 is an electrical contact point. In some embodiments, the contact point 106 is at least partially electrically conductive. In some embodiments, the contact point 106 is a stand-alone component. For example, the contact point 106 may be coupled to but separate from the rest of the DNA protection system 102. In other embodiments, the contact point 106 is integrated with a surface or structure of another component or system.

In some embodiments of the DNA protective system, grounded conductive elements capable of sustaining an ESD event may be insulated to prevent untuned discharge. For example, metal screws may be replaced with tuned shunting screws or insulated components, facias and other body panels of different systems may be swapped for tuned or insulating equivalents. Some metallic components may not be grounded but still facilitate an ESD event (door knob, light switch plate, etc.). DNA protection systems may be put in place which equalize the charge across the user and environmental component without causing an ESD event.

In some cases, conductive elements may not be fully insulated. In these situations, additional shunting considerations or calculations may be employed as protection. In one example, an ESD event may occur on a bedsheet as a sleeper moves around in the bed. A tuned charge shunt path may drain off the charge as it is generated and reduce the chance of an ESD event. In another example, when two people are sleeping in a single bed, tuned shunting paths may be formed in bedding, clothing, and other components to constantly or periodically shunt charge to prevent a mutual ESD event between the sleepers and ESD events between a sleeper and other points of contact such as an alarm clock, bedframe, bedside lamp, light switch, and the like.

The resistive element 108 is electrically coupled to the contact point 106. The resistive element 108 may be a fixed or variable resistive component. For example, the resistive element 108 may be manually or automatically adjustable. In some embodiments, the resistive element 108 is coupled to a sensor or other component which adjusts the resistivity of the resistive element 108 based on a detected condition. For example, a detected charge on a biological system 104 at or near the contact point 106 may allow the resistive element 108 to be tuned to a corresponding resistivity.

In the illustrated embodiment, the resistive element 108 is a separate component from the contact point 106. In some embodiments, the resistive element 108 is integrated with the contact point 106. For example, the resistive element 108 may be a material that makes up at least a portion of the contact point 106 or a component integrated into or coupled to the contact point 106.

In some embodiments, the resistive element 108 has a bulk resistance in the giga-ohm range. In other embodiments, the resistive element 108 has a bulk resistance that is magnitudes higher or lower than the giga-ohm range. The resistance value of the resistive element 108 may be tuned to correspond to a time of contact between the biological system 104 and the contact point 106.

The resistive element 108 may also be tuned based on the intrinsic material properties of the materials involved in the charge path. For example, the resistance of the DNA protection system 102 may be tuned based on an expected contact time. The contact time may be less than five seconds, less than one second, or a few milliseconds. In some embodiments, the resistive element 108 is tuned to correspond to five time-constants. Other embodiments may be tuned to few or more than five time-constants.

In some embodiments, the resistive value of the resistive element 108 is determined based on a factor applied to a time-constant for the material or object involved as well as the desired percentage or amount of charge to shunt or drain during the period of contact between the biological system 104 and the electrical contact point 106.

In some embodiments, the resistive element 108 is coupled to the connection point 110. The connection point 110 may be a fixed or mobile ground. The connection point 110 may form a circuit ground or earth ground. The connection point 110 may be part of an energy storage system or a true ground.

In some embodiments, the connection point 110 is not a true ground but representative of the other side of a potential ESD event. For example, if a person is exiting an automobile after building up a significant amount of charge with respect to the automobile, a tuned DNA protective system 102 may shunt the charge between the person and the automobile during the exit at a rate that is slow enough, and to a voltage level low enough, to avoid an ESD event. Because the automobile is on insulating rubber tires, no true ground is present in the system. As described above, the rate of the charge transfer should be tuned such that the transfer does not reach a rate which could potential damage DNA.

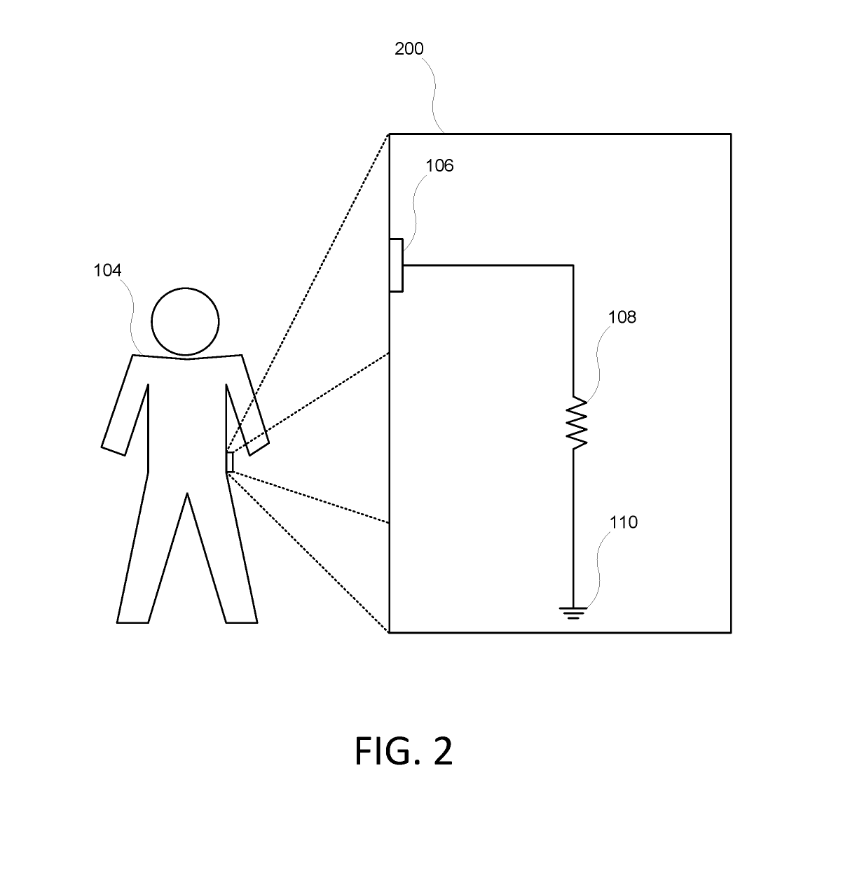

FIG. 2 is an enlarged view illustrating one embodiment of a DNA protective apparatus 200 local to a biological system 104 in accordance with the present disclosure. In the illustrated embodiment, the DNA protective apparatus 200 is proximal the biological system 104. In some embodiments, the DNA protective apparatus 200 is small relative to the biological system 104. The DNA protective apparatus 200 may be contained, as shown, in a single unit or separated into two or more units.

One or more of the components of the DNA protective apparatus 200 may be as described above with respect to FIG. 1. For example, one or more of the contact point 106 and the resistive element 108 may be configured or tuned for an expected duration of contact, by the biological system 104, at the contact point 106. In some embodiments, the DNA protective apparatus 200 is in frequent contact with the biological system 104.

In other embodiments, the DNA protective apparatus 200 is in infrequent contact with the biological system 104. In some embodiments, the DNA protective apparatus 200 initiates a charge drain or shunting. The shunting may be initiated via actuation of some component of the DNA protective apparatus 200 to cause contact or complete a circuit with the biological system. Shunting may also be initiated by providing a notification or alert to provide contact with the DNA protective apparatus 200. In other embodiment, the DNA protective apparatus 200 is passive in that the DNA protective apparatus 200 may shunt charge in response to contact or other input from the biological system 104 without the DNA protective apparatus 200 executing processes to initiate the shunting.

In some embodiments, the DNA protective apparatus 200 is associated with a single biological system 104. In other embodiments, the DNA protective apparatus 200 is associated with a plurality of biological systems 104. For example, the DNA protective apparatus 200 may be a personal device or a public or common device. In some embodiments, the DNA protective apparatus 200 is a satellite unit that is associated with a base unit. In other embodiments, the DNA protective apparatus 200 is a stand-alone unit.

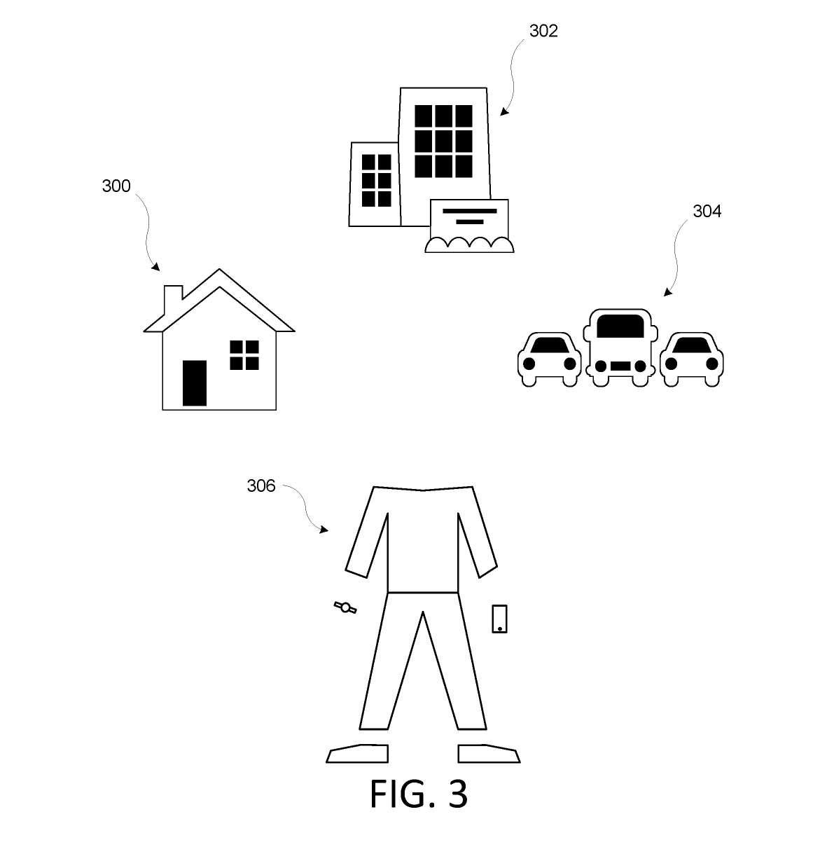

FIG. 3 is a diagram illustrating different environments in which embodiments of DNA protection may be implemented in accordance with the present disclosure. The illustrated embodiment includes a domicile environment 300. The domicile environment 300 may include a home, hotel, dormitory, or other residential or commercial domicile. The illustrated embodiment also includes an occupational environment 302. The occupational environment 302 may include an office, shop, meeting place, restaurant, store, or the like.

Each of the domicile environment 300 and the occupational environment 302 may DNA protective systems 102 or apparatuses 200. In these environments 300 and 302, the DNA protective systems 102 may be placed or integrated into human interfaces or other systems which are contacted by a biological system 104. For example, the protective systems 102 may include one or more of carpeting, rugs, tile, laminate, wood, and other flooring or floor coverings, light switches, doors, door plates, door handles, hand rails, faucets, toilets, furniture such as beds and bedding/sheets, tables, chairs, desks, elevators, elevator call buttons, stairs and stair coverings, shopping carts and bags, registers, shelves, displays, keypads, pens, styluses, keyboards (and keyboard keys), mice, touchpads, computers such as laptops, tablets, and desktops, and other known systems, structures, and the like.

FIG. 3 also includes a transportation environment 304. The transportation environment 304 may include a can, bus, train, boat, plane, and the like. Embodiments described herein may be incorporated into human interfaces or other systems and structures of the transportation environment 304 which comes into contact with a biological system 104. For example, the DNA protective system 104 may include one or more of doors, door handles, door knobs, door panels, keypads, key slots, kickplates, vehicle body panels, vehicle interior panels, steering wheels or other control interfaces such as parking breaks, shift handles, pedals, stereo and other entertainment controls, and climate controls, and the like, seats, flooring, consoles, dashes, seatbelts, seatbelt receivers, and other known systems, structures, and the like.

The illustrated embodiment of FIG. 3 also includes a personal environment 306. The personal environment 306 may include clothing. For example, socks, shoes, and other footwear may be effective. The personal environment 306 may also include accessories such as jewelry, belts, hats, purses, wallets, keychains, glasses, and the like. The personal environment 306 may also include personal devices such as phones, tablets, watches, music players, headphones, and the like.

In some embodiments, the personal devices may track electrostatic buildup on the biological system 104 and provide a warning or alert in response to the electrostatic buildup reaching unsafe levels. The personal device may prompt a discharge or provide other instructions to facilitate safe and non-damaging charge shunting. Additionally, the personal device or other systems may track charge levels, ESD events, and the like and provide records or data in a feedback loop to the user to modify behavior, address potential hazards, or the like.

In some embodiments, one or more of the components of the environments 300, 302, 304, and 306 cooperate to drain or shunt electrostatic charge from a biological system 104 at a rate that reduces the chance of damage to DNA of the biological system 104 due to electrostatic discharge events. For example, shoes may be tuned to work in tandem, as a system, with tuned carpets or other flooring surfaces/materials.

While some embodiments incorporate DNA protective systems 102 and apparatuses 104 into existing components, other embodiments provide DNA protective systems 102 and apparatuses 104 as standalone components which may be hidden or openly identifiable. In some embodiments, a presence of the DNA protective systems 102 and apparatuses 104 may be masked or hidden to preserve aesthetic characteristic of the corresponding structure. In other embodiments, the presence of the DNA protective systems 102 and apparatuses 104 may be emphasized or highlighted to prompts contact or use of the DNA protective systems 102 and apparatuses 104.

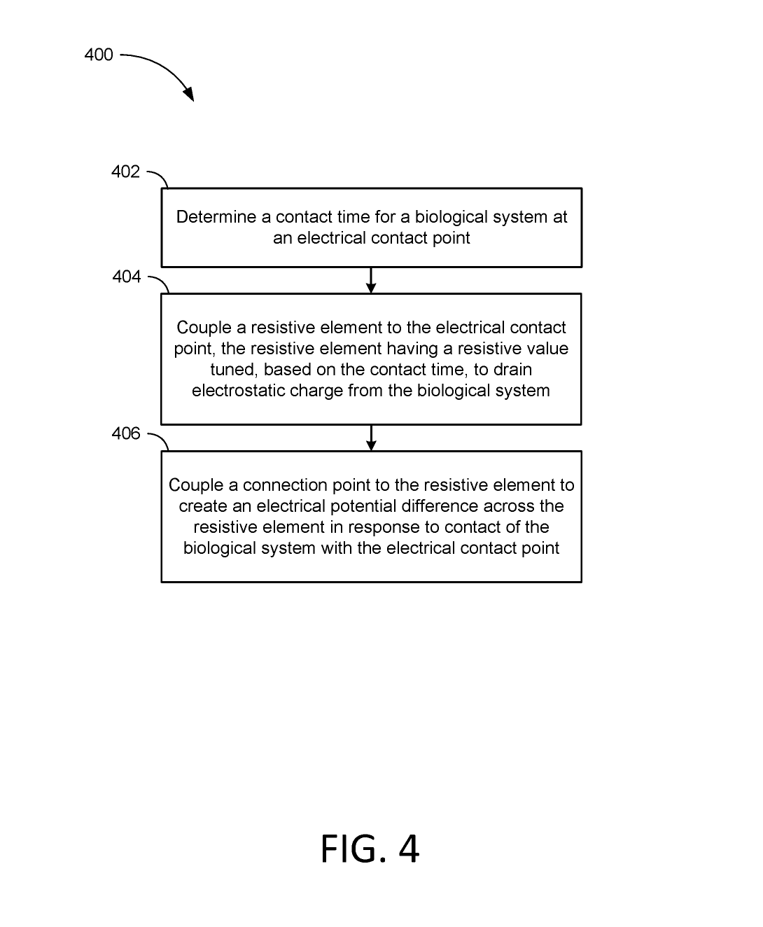

FIG. 4 is a flowchart diagram illustrating one embodiment of a method 400 for protecting DNA from electrostatic discharge damage in accordance with the present disclosure. In the illustrated embodiment, the method 400 includes determining 402 a contact time for a biological system at an electrical contact point. The method 400 also includes coupling 404 a resistive element to the electrical contact point, the resistive element having a resistive value tuned, based on the contact time, to drain electrostatic charge from the biological system. The method 400 also includes coupling 406 a connection point to the resistive element to create an electrical potential difference across the resistive element in response to contact of the biological system with the electrical contact point.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus to protect a biological system from molecular damage due to electrostatic discharge, the apparatus comprising:
an electrical contact point positioned to facilitate electrical communication with the biological system;
a resistive element coupled to the electrical contact point, the resistive element having an electrical resistive value tuned to drain electrostatic charge from the biological system based on a contact time of the electrical contact point with the biological system; and
a connection point coupled to the resistive element to create an electrical potential difference across the resistive element in response to the contact of the electrical contact point with the biological system.

2. The apparatus of claim 1, wherein a duration of the contact of the electrical contact point with the biological system is less than approximately one second.

3. The apparatus of claim 1, wherein a duration of the contact of the electrical contact point with the biological system is less than approximately five seconds.

4. The apparatus of claim 1, wherein the electrical contact point is disposed in or on a building.

5. The apparatus of claim 4, wherein the electrical contact point is disposed in or on a fixture of the building.

6. The apparatus of claim 4, wherein the electrical contact point is disposed in or on a human interface of an item within the building.

7. The apparatus of claim 4, wherein the electrical contact point is disposed in or on a human interface of a component of the building.

8. The apparatus of claim 1, wherein the electrical contact point is disposed in or on a human interface of a vehicle.

9. The apparatus of claim 8, wherein the human interface of the vehicle comprises at least one of a list of components, the list comprising a steering wheel, a seat, a seat belt, a door handle, a door panel, a shift knob, an entertainment control, a climate control, and a floor covering.

10. The apparatus of claim 1, wherein the connection point is in electrical communication with a surface comprising a potential electrostatic discharge site.

11. A method for protecting a biological system from electrostatic discharge damage, the method comprising:
determining a contact time for the biological system at an electrical contact point;
coupling a resistive element to the electrical contact point, the resistive element having a resistive value tuned, based on the contact time, to drain electrostatic charge from the biological system; and
coupling a connection point to the resistive element to create an electrical potential difference across the resistive element in response to contact of the biological system with the electrical contact point.

12. The method of claim 11, further comprising determining the resistive value based on a characteristic of a material of at least one of the electrical contact point and the connection point.

13. The method of claim 12, wherein the characteristic comprises a breakdown voltage of the material.

14. The method of claim 12, wherein the characteristic comprises a resistivity of the material.

15. A wearable system to protect a biological system from molecular damage due to electrostatic discharge, the wearable system comprising:
an electrical contact point positioned to facilitate electrical communication with the biological system;
a resistive element coupled to the electrical contact point, the resistive element having an electrical resistive value tuned to drain electrostatic charge from the biological system based on a contact time of the electrical contact point with the biological system;
a connection point coupled to the resistive element to create an electrical potential difference across the resistive element in response to the contact of the electrical contact point with the biological system; and
a tracking system to track data corresponding to the biological system.

16. The wearable system of claim 15, wherein the data tracked by the tracking system comprises a charge level of the biological system.

17. The wearable system of claim 16, further comprising an alert generated in response to the charge level reaching an unsafe level.

18. The wearable system of claim 15, wherein the data tracked by the tracking system comprises a number of discharge events involving the biological system.

19. The wearable system of claim 15, wherein the wearable system is a passive system.

20. The wearable system of claim 15, wherein the wearable system is an active system.
